(19) United States
(12) Patent Application Publication
(10) Pub. No.: US 2021/0356428 A1
(43) Pub. Date: Nov. 18, 2021

(54) Rapid Virus Detection

(71) Applicant: Slobodan Petrovic, Happy Valley, OR (US)

(72) Inventor: Slobodan Petrovic, Happy Valley, OR (US)

(21) Appl. No.: 17/321,369

(22) Filed: May 14, 2021

Related U.S. Application Data
(60) Provisional application No. 63/024,755, filed on May 14, 2020.

(51) Int. Cl. G01N 27/48

(52) U.S. Cl. CPC G01N 27/48

(57) ABSTRACT

A electrochemical detection cell for rapid detection of viruses in a mucous sample. The detection cell has a potentiostat, a working electrode, a counter electrode, and electrolyte. When the mucous sample is dissolved in the electrolyte the detection cell is capable of measuring the presence of a specific virus that was present in the mucous sample.

### CROSS-REFERENCES TO RELATED APPLICATION

This application claims the benefit of U.S. Provisional Patent Application No. 63/024,755, filed May 14, 2020, which is incorporated by reference herein in its entirety.

### BACKGROUND

Viruses, including corona viruses, enter a person's system through the mouth or nose and reside in the mucus of the mouth and nose. They can be detected by taking a sample of the mucus using a swab, placing the sample in a suitable electrolyte, and measuring the concentration using electrodes and a device described as a potentiostat. A potentiostat is a versatile power supply capable of applying current and voltage in a variety of ways for maximum selectivity of the measurement.

Viruses can also contaminate a person's skin particularily a person's hands. The ability to detect the virus and treat it, if present, in a continuous fashion, with minimal or no effect on the person is a preferable alternative to constant washing and disinfection.

### SUMMARY

Viruses, such as COVID-19 can be detected using an electrochemical device. Two devices are disclosed here. First, a device is disclosed that can detect viruses in a mucus sample. A swab is used to collect a mucus sample from person's mouth or nose and dissolving it a solution or electrolyte. Next, the sample of electrolyte containing the virus is inserted in a cell that contains electrolyte and two or three probes, called electrodes. Alternatively, one can insert the swab directly into electrolyte in the cell. One of the electrodes of the cell is the working electrode, the second one is counter and the third one is the reference electrode. The reference electrode is optional and can improve detection accuracy. The working electrode can be a metallic electrode, or an electrode with a modified surface, designed with receptors for attachment and selective detection of a certain virus. The measurement is commonly conducted by applying voltage and measuring current, but other variations of current and voltage can be used. The amount of current depends on the concentration of viruses. The entire measurement can be shorter than 30 seconds. Different viruses can be detected in this way by varying the electrical signal (i.e., voltage) and/or modified layer on the electrode surface. In addition to a method where current is measured, viruses can be identified through electrochemical impedance spectroscopy.

Alternatively, the swab can be made with the electrode at the tip and after taking the sample from a person it will be directly immersed in the cell, already containing electrolyte, counter and reference electrode. The cell can be made conveniently portable by having a small potentiostat attached to it. When the working electrode probe with the sample of the mucus on the tip is inserted into the cell and immersed in the electrolyte the measurement can proceed.

The concept of detecting viruses with a potentiostat can also be applied to detecting and treating viruses such as COVID-19 on person's skin. The measurement can predominantly concentrate on one or more areas on the skin. A small patch is applied that contains a battery and a device to apply and control voltage, while two probes are touching the thin layer of moisture on the skin. The probes are passivated on the side touching the tissue to prevent any electrical current going through the skin. Instead, the ionic current is established through the moisture on the skin. A suitable gel can be additionally applied to the skin, hands for example, to increase the moisture. Any virus present on the skin can travel through the layer of moisture and reach electrodes where it can be detected and subsequently deactivated by decomposing proteins, which form the outer layer of the virus. The device can: 1) provide instantaneous detection of the virus present in the moisture on the skin, and 2) deactivate the virus during prolonged treatment.

### DESCRIPTION OF THE DRAWINGS

FIG. 1A is a schematic of a swab.

FIG. 1B is a schematic of swab with an electrode.

FIG. 2A is a schematic of a detection cell used with a swab.

FIG. 2B is a schematic of a detection cell used with a swab having an electrode.

FIG. 3 is a schematic of a virus dectection device for measuring and treating viruses on the skin.

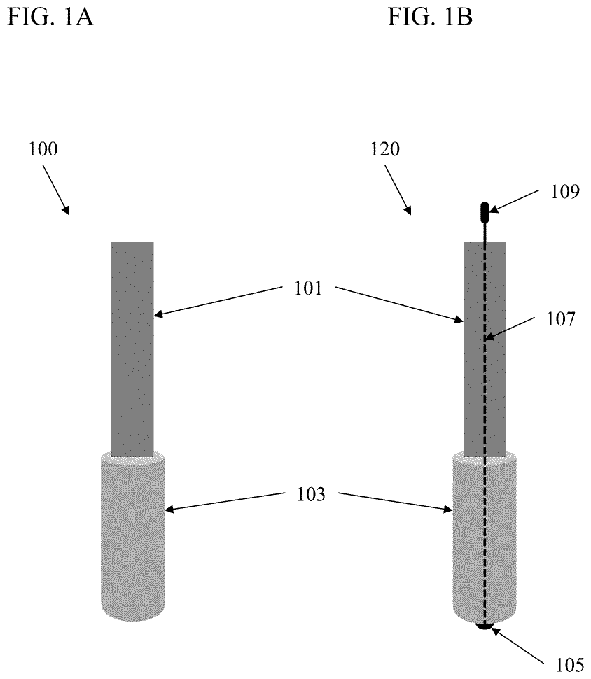

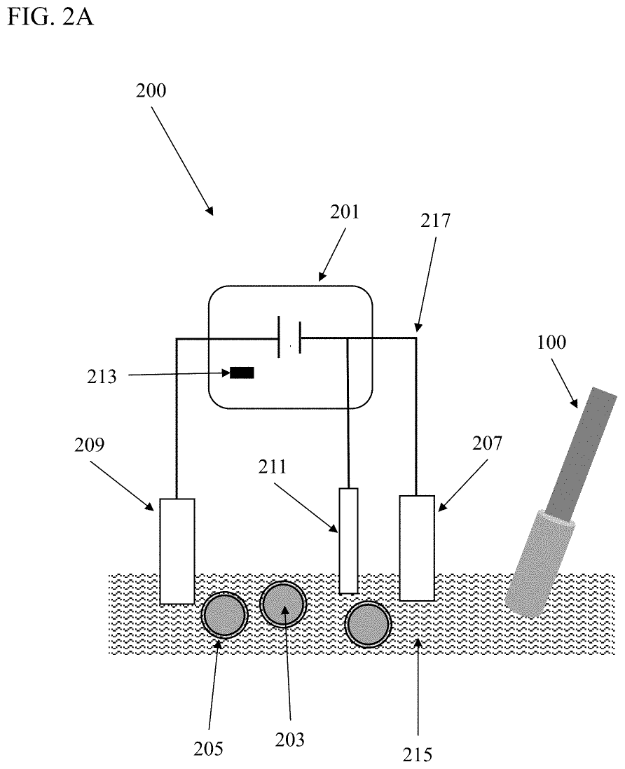

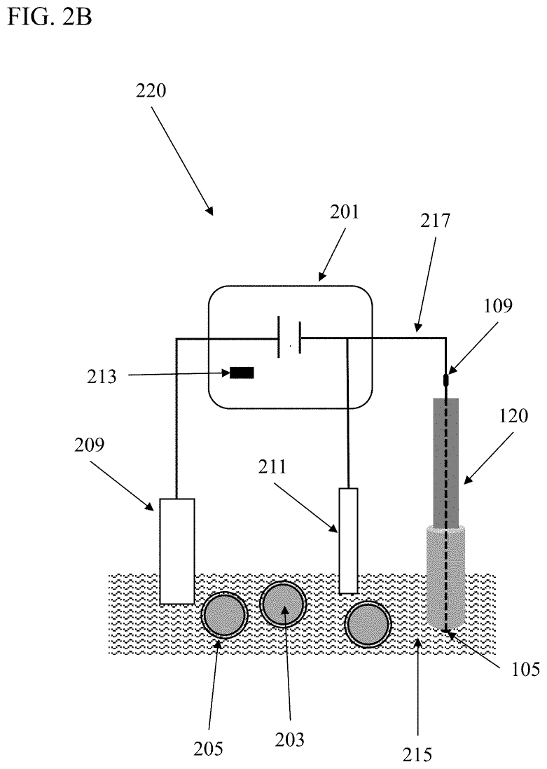

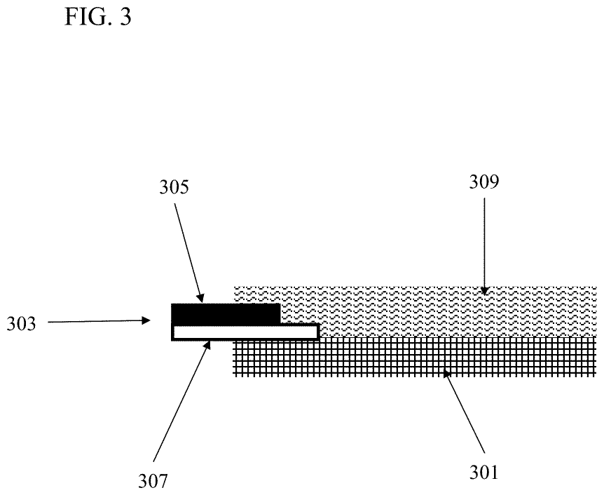

### DESCRIPTION

A swab 100 consists of a member 101 and absorbant material 103. A conductive swab 120 also has a member 101 and absorbant material 103, and includes a probe 105, a wire 107, and a connector 109. Either the swab 100 or the conductive swab 120 may be used to collect a sample of mucus from the nose or mouth. When used in the nose or the mouth, the sample of mucus is captured on the absorbant material 103 of a swab 100 or conductive swab 120 and can be transferred to the detection cell.

Detection cell 200 is for use with a swab 100. The detection cell has a measurement device 201 which has a potentiostat and is preferably powered by a battery. The type of battery in the measurement device 101 can be, but no limited to, a primary battery (examples include ZnO, Leclanche, alkaline, lithium polymer, and lithium ion) with the option to easily replace the battery. Alternatively, a rechargeable battery can be used, such as nickel metal hydride or lithium polymer, along with an appropriate mechanism for recharging the battery. Alternatively, the measurement device 201 can be powered by 110 V wall outlet with an appropriate transformer suitable for the measurement device 201.

The potentiostat in the measurement device 201 prefereably has a working electrode 207, a counter electrode 209, and a reference electrode 211 which are each in contact with electrolyte 215 in the detection cell 200 through wires 217. A reference electrode 211 permits a potentiostat to more accurately control the voltage.

The sample of mucus from the swab 100 is inserted into the electrolyte 215 of the detection cell 200. If there are virus cells in the mucus sample they move through the electrolyte 215 and can be measured by the measurement device 201.

Viruses 203, including corona viruses, have a protein skin 205 that will cause the virus to diffuse to the working electrode 207. As a result of the voltage applied by the potentiostat of the measurement device 201, a small ionic current is created in the electrolyte 215 between the working electrode 207 and the counter electrode 209. The ionic current drives the viruses 203 towards the working electrode 207 through diffusion, convection, and migration. Upon arrival at the working electrode 207, the protein skin 205 of the virus reacts at the working electrode 207 at a certain voltage, resulting in generation of current, which is used to determine the presence of the virus 203 and its concentration.

Preferably, the voltage of the potentiostat of the measurement device 201, can be adjusted. Adjustment of the voltage may be useful in targeting particular viruses of concern that are, or may be, in the sample of the mucus that is inserted into the electrolyte 215. It is possible to calibrate the testing system for a certain virus, or to use electrodes with modified surfaces that contain layers sensitive to a particular virus. It is furthermore possible to detect multiple viruses in a single sample by varying the voltage. The potentiostat may configured with a manual dial mounted on detection cell or remotely using a wireless transceiver such as a Bluetooth transceiver 213 to connect with a smart phone or other computing device with an application designed to interface with the detection cell 200.

An alternative embodiment of a detection cell 220 is configured with a connector that can connect to the connector 109 of a conductive swab 120. The conductive swab 120 with a sample of mucus is inserted into the electrolyte of the detection cell 220. If there are virus cells in the mucus sample they transfer to the electrolyte 215 and can be measured by the measurement device 201. The conductive swab 120 becomes the working electrode of the measurement device 201.

As a result of the voltage applied by the potentiostat of the measurement device 201, a small ionic current is created in the electrolyte 215 between the conductive swab 120 now functioning as a working electrode and the counter electrode 209. The ionic current drives the viruses 203 towards the conductive swab 120 through diffusion, convection, and migration. Upon arrival at the probe 105 of the conductive swab 120, the protein of the virus reacts at the probe 105 at a certain voltage, resulting in generation of current, which is used to determine the presence of the virus 203 and virus concentration.

The metal probes of the conductive swab 120, working electrode 207, counter electrode 209, and reference electrode 211 can be made a number of metals such as gold, palladium, copper, or silver. They can also be modified using special compounds containing receptors for particular proteins designed to more readily enable electron transfer to the virus protein 205. The shape of the electrodes can typically be a cross-section of a wire or a cylinder, embedded in a plastic housing. However, any other shape of the electrode is possible. The radius of a sphere exposed to measured solution can typically be from 0.1 to 20 mm.

The metal probes can also be modified to include rotation using a small motor for faster convection of the viruses to the electrode surface.

The whole assembly, a cell with electrodes and electrolyte and the potentiostat can be made into a portable device for easy carrying and rapid testing capabilities. Multiple measurements may be made by having an additional reservoir, as part of a kit, with a cleaning solution for the probe that is used as a swab.

A device for detection of viruses on skin has a shape of patch to be attached to the skin with a variety of non-permanent methods. The patch contains a thin-form factor battery and electronics containing a potentiostat and electrical probes, i.e., electrodes 303. Each electrode 303 is electrically insulated using an insulator 307 on the side that presses into the skin 301, while the metal contact 305 is exposed to the moisture 309 on the skin. Skin moisture 309 serves as an electrolyte for the electrochemical circuit, which enables viruses to be transported to the working electrode, where they get first detected and with a prolonged use, destroyed.

The metal probes 305 can be made a number of metals such as gold, palladium, copper, or silver. An electrode 305 can also be modified using special compounds containing receptors for particular proteins designed to more readily enable electron transfer to the virus protein. The preferred shape of the electrodes is flat to conform with the shape of one's skin. However, any other shape of the electrode 305 is possible. The size can typically be from 0.1 to 20 mm.

The ionic conductivity of the skin moisture 309 can be enhanced by rubbing a gel type electrolyte or a moisturizer into the skin. This would enable easier transport of the viruses to the electrodes.

The device is completely safe as there is no electrical current applied to the skin. It can be used for an instantaneous detection of the viruses and if necessary for a prolonged treatment to destroy the viruses or to prevent transmission through touch.

What is claimed is:

1. An apparatus comprising:
a swab for obtaining a mucus sample;
a detection cell having, a potentiostat, a working electrode, a counter electrode, a reference electrode, and electrolyte;
whereby the detection cell is capable of detecting viruses in the mucus sample.

2. The apparatus of claim 1 further comprising:
a wireless transceiver;

3. The apparatus of claim 1 further comprising:
said potentiostat having adjustable voltage.

4. An apparatus comprising:
A conductive swab for obtaining a mucus sample with a connector;
a detection cell having, a potentiostat, a counter electrode, a reference electrode, electrolyte and a second connector;
said conductive swab electrically connectiable to said detection cell to form a working electrode for the detection cell;
whereby the detection cell is capable of detecting viruses in the mucus sample.

5. The apparatus of claim 4 further comprising:
a wireless transceiver.

6. The apparatus of claim 4 further comprising:
said potentiostat having adjustable voltage.
